(19) United States
(12) Patent Application Publication
(10) Pub. No.: US 2021/0400963 A1
(43) Pub. Date: Dec. 30, 2021

(54) Multi-Use Fermentation Products Obtained Through Production of Sophorolipids

(71) Applicant: Locus IP Company, LLC, Solon, OH (US)

(72) Inventors: Sean FARMER, Ft. Lauderdale, FL (US); Ken ALIBEK, Solon, OH (US)

(21) Appl. No.: 17/280,882

(22) PCT Filed: Sep. 26, 2019
(86) PCT No.: PCT/US2019/053226

§ 371 Date: Mar. 27, 2021

Related U.S. Application Data
(60) Provisional application No. 62/738,504, filed on Sep. 28, 2018.

(51) Int. Cl. A01N 43/16; A01N 63/00; A23K 20/163; A23L 31/10; A61K 9/127

(52) U.S. Cl. CPC A01N 43/16; A01N 63/00; A61K 9/127; A23L 31/10; A23K 20/163

(57) ABSTRACT

The subject invention provides methods for cultivating Starmerella bombicola yeasts for sophorolipid production, as well as a yeast fermentation product comprising supernatant, yeast cell biomass and other residual growth by-products that result when the sophorolipids are harvested. Methods of using this yeast fermentation product in animal feed, bioremediation, mining and/or bioleaching, agriculture, oil and gas recovery, human health, and many other applications, are also provided.

### CROSS-REFERENCE TO RELATED APPLICATION

This application claims priority to U.S. Provisional Patent App. No. 62/738,504, filed Sep. 28, 2018, which is incorporated herein by reference in its entirety.

### BACKGROUND OF THE INVENTION

Cultivation of microorganisms such as bacteria, yeast and fungi is important for the production of a wide variety of useful bio-preparations. One area in which microorganisms are particularly useful is in the production of biosurfactants, which are a structurally diverse group of surface-active substances that can be used in, for example, food industries, oil and gas recovery, agriculture, mining, environmental remediation, and waste management.

All biosurfactants are amphiphiles consisting of two parts: a polar (hydrophilic) moiety and non-polar (hydrophobic) group. Due to their amphiphilic structure, biosurfactants increase the surface area of hydrophobic water-insoluble substances, increase the water bioavailability of such substances, and can change the properties of bacterial cell surfaces.

Biosurfactants include low molecular weight glycolipids (e.g., rhamnolipids, sophorolipids, mannosylerythritol lipids and trehalose lipids), lipopeptides (e.g., surfactin, iturin, fengycin and lichenysin), flavolipids, phospholipids, and high molecular weight polymers such as lipoproteins, lipopolysaccharide-protein complexes, and polysaccharide-protein-fatty acid complexes. The common lipophilic moiety of a biosurfactant molecule is the hydrocarbon chain of a fatty acid, whereas the hydrophilic part is formed by ester or alcohol groups of neutral lipids, by the carboxylate group of fatty acids or amino acids (or peptides), organic acid in the case of flavolipids, or, in the case of glycolipids, by the carbohydrate.

These advantageous molecules can be particularly useful in the development of environmentally-friendly technology, given that they are biodegradable and can be produced efficiently using selected organisms on renewable substrates. Most biosurfactant-producing organisms produce biosurfactants in response to the presence of a hydrocarbon source (e.g., oils, sugar, glycerol, etc.) in the growing media. A variety of microorganisms such as bacteria, fungi, and yeasts can produce biosurfactants, such as, for example, Arthrobacter spp.; Bacillus spp. (B. subtilis, B. amyloliquefaciens, B. pumillus, B. cereus, B. licheniformis); Candida spp. (C. albicans, C. rugosa, C. tropicalis, C. lipolytica, C. torulopsis); Campylobacter spp.; Cornybacterium spp.; Flavobacterium spp.; Pichia spp.; Pseudomonas species (P. aeruginosa, P. putida, P. florescens, P. fragi, P. syringae); Rhodococcus spp.; Rhodotorula spp.; Starmerella spp.; Wickerhamiella spp., Wickerhamomyces spp. and so on.

In particular, the non-pathogenic yeast Starmerella bombicola, or Candida bombicola, and other yeast species such as Candida apicola, Candida batistae, Rhodotorula bogoriensis and Wickerhamiella domnericqiae, are known for their ability to produce a specific class of glycolipids known as sophorolipids (SLP) during the stationary phase of fermentation. The structure of SLP comprises sophorose, consisting of two glucose molecules, linked to a fatty acid by a glycosidic ether bond. SLP are categorized into two general forms: the lactone form in which the carboxyl group in the fatty acid side chain and the sophorose moiety form a cyclic ester bond; and the acidic (linear) form in which the bond is hydrolyzed.

S. bombicola are oleaginous yeast species, meaning they can utilize oleaginous substrates such as alkanes and oils as carbon sources, and can handle those substrates in relatively high concentrations. Moreover, S. bombicola can produce SLP in high amounts, which are excreted into the fermentation medium.

Two principle forms of microbe cultivation exist: submerged cultivation and solid state cultivation. Bacteria, yeasts and fungi can all be grown using either the solid state or submerged cultivation methods. Both methods require a nutrient medium for microbial growth, which can either be in a liquid form for submerged cultivation, or a solid form for solid state cultivation. Typically, the nutrient medium includes a carbon source, a nitrogen source, salts and appropriate additional nutrients and microelements. The pH and oxygen levels are maintained at values suitable for a given microorganism.

In submerged fermentation of S. bombicola for SLP production, an oil such as, e.g., canola oil, can be used as a hydrophobic carbon source. The resulting product is a highly viscous, brown-colored layer of SLP that can be separated from the culture medium and, if desired, processed and/or purified for various uses. What remains is a product containing leftover fermentation broth and yeast cell biomass, which is often discarded, unused, as waste.

There exists an enormous potential for the use of microbes and their growth by-products, in a broad range of industries. However, current methods of producing microbe-based products are often inefficient and difficult to scale for industrial applications. The use of biological agents in industry has been greatly limited by difficulties in production, transportation, administration, pricing and efficacy. This problem is exacerbated by losses in viability and/or activity due to processing, formulating, storage, and stabilizing prior to distribution.

Furthermore, when a microbial metabolite is extracted from the products of fermentation, large quantities of valuable product are often left behind and discarded, resulting in large quantities of unused, potentially valuable product. Thus, methods are needed for efficiently producing microorganisms and microbial metabolites on a large scale, as well as methods of minimizing the amount of wasted product that results from harvesting metabolites, such as sophorolipids, from the fermentation medium.

### BRIEF SUMMARY OF THE INVENTION

The subject invention provides microbe-based compositions, as well as methods for producing them. Methods are also provided for using these compositions in a variety of applications, including animal feed, bioremediation, mining and/or bioleaching, agriculture, oil and gas recovery, human health, and many others.

Specifically, the subject microbe-based compositions are yeast fermentation products obtained during production of biosurfactants, such as, e.g., sophorolipids (SLP). Through submerged cultivation of a biosurfactant-producing microorganism, biosurfactants are excreted into the fermentation broth and then harvested for further processing and/or purification. What remains in the culture is a product with several different utilities. Advantageously, when produced according to the subject invention, recycling of these leftover culture products can reduce waste and increase the profitability of biosurfactant production—particularly for large-scale, commercial operations.

In certain embodiments, the subject invention provides a yeast fermentation product comprising supernatant and, optionally, yeast cell biomass, resulting from cultivation of a yeast microbe. Preferably, the yeast is a biosurfactant-producing yeast that has been cultivated for the purpose of producing a biosurfactant. Even more preferably, the yeast is Starmerella bombicola, which is capable of producing sophorolipid (SLP) biosurfactants in high concentrations.

In one embodiment, the supernatant of the yeast fermentation product comprises yeast growth by-products, such as, e.g., excreted metabolites and/or cell wall components. In some embodiments, the growth by-product is a residual biosurfactant.

In certain embodiments, the yeast fermentation products according to the subject invention can be superior to, for example, purified microbial metabolites alone, due to, for example, the advantageous properties of yeast cell walls. These properties include high concentrations of mannoprotein, as well as the biopolymer beta-glucan as a part of a yeast cell wall's outer surface. These compounds can serve as, for example, effective emulsifiers. Additionally, the yeast fermentation product further can comprise residual biosurfactants in the culture, as well as other metabolites and/or cellular components, such as solvents, acids, vitamins, minerals, enzymes and proteins. Thus, the yeast fermentation products can, among many other uses, act as biosurfactants and can have surface/interfacial tension-reducing properties.

The subject invention further provides methods for producing the subject yeast fermentation product, wherein the method comprises cultivating a yeast in culture medium using a submerged fermentation system, wherein the yeast produces a biosurfactant into the medium; and harvesting the biosurfactant to leave behind a supernatant and yeast cell biomass in the fermentation system. The supernatant and yeast cell biomass that are left behind comprise the yeast fermentation product.

Preferably, the yeast according to the subject method is a biosurfactant-producing yeast. Even more preferably, the yeast is Starmerella bombicola, which is capable of producing sophorolipid (SLP) biosurfactants in high concentrations. In a specific embodiment, cultivation of the yeast occurs at 25-28° C. for 1 to 10 days. In one embodiment, allowing the culture to settle after cultivation produces a layer of SLP sediment in the culture comprising about 10-15% SLP, or about 4-5 g/L. Advantageously, once the SLP layer is harvested from the culture, about 1-4 g/L of SLP can still remain in the yeast fermentation product, as well as other advantageous yeast growth by-products and cellular components.

In specific embodiments of the subject methods, the culture medium can be formulated for enhanced biosurfactant production. For example, in one embodiment, the medium can comprise yeast extract (e.g., Saccharomyces cerevisiae autolysates and/or hydrolysates), glucose or another carbon source, and urea or another ammonium source. In another embodiment, more than one carbon source can be utilized, wherein at least one of the carbon sources is an oil, such as, e.g., canola oil.

In some embodiments, the methods can be used for producing a biosurfactant product, wherein, after harvesting the SLP sediment layer from the culture, the method comprises processing and/or purifying the SLP, if desired. In one embodiment, the SLP are left in a crude and/or unpurified form. The crude form can comprise from about 0.001% to about 99%, about 25% to about 75%, about 30% to about 70%, about 35% to about 65%, about 40% to about 60%, about 45% to about 55%, or 50% pure biosurfactant in liquid broth.

In certain preferred embodiments, the fermentation systems utilized according to the subject methods are scalable, distributable fermentation systems (also referred to herein as “systems,” “reactors,” “reactor systems,” and/or “units”). In one embodiment, the methods utilize reactors that, in addition to growing biosurfactant-producing yeasts, can be used to grow other microbes, including fungi and bacteria.

Advantageously, the subject systems can be utilized on a small scale (e.g., in a lab setting) or a large and/or industrial scale (e.g., for agriculture). The subject systems and cultivation methods not only substantially increase the yield of microbial products per unit of nutrient medium but simplify production and facilitate portability. Advantageously, the method and equipment of the subject invention reduce the capital and labor costs of producing microorganisms and their metabolites on a desired scale.

In certain embodiments, methods for using the subject yeast fermentation product are provided, wherein the methods comprise applying the yeast fermentation product to a target application site. The product can be harvested from the fermentation system and applied directly to the target site. In other embodiments, the product is stored and/or transported after harvesting and prior to application to the target site.

In one embodiment, the yeast fermentation product can be used without further modification, meaning that it can be taken from the fermentation system in which it was produced and applied, as is, directly to the target site.

In one embodiment, the yeast fermentation product can be modified and/or formulated for a particular use after harvesting. For example, the product can be concentrated or diluted, dried, and/or mixed with additional ingredients, as is deemed necessary for a particular application. Furthermore, the yeasts of the composition can be in an active or inactive form.

In one exemplary embodiment, the yeast fermentation product can be used as a nutritional supplement for an animal, wherein the target application site is an animal's feed and/or drinking water.

In one exemplary embodiment, the yeast fermentation product can be used as a bioremediation agent, wherein the target application site is a site, such as water or soil, that has been contaminated with, for example, oil from an oil spill, or a toxin, such as arsenic.

In one exemplary embodiment, the yeast fermentation product can be used as a mining and/or bioleaching agent, wherein the target application site is ore and/or coal that contains one or more valuable minerals or elements.

In one exemplary embodiment, the yeast fermentation product can be used for enhanced agricultural production, wherein the target application site is soil, a plant and/or a seed.

In one exemplary embodiment, the yeast fermentation product can be used for enhanced oil recovery (EOR), wherein the target application site is an oil and/or gas well, a subterranean formation, and/or any piece of equipment associated with oil and/or gas recovery, transport, refining, and processing.

In one exemplary embodiment, the yeast fermentation product can be used as an adjuvant composition and/or as a digestive aide in humans, wherein the target site is a pharmaceutical, a supplement, a nutrient and/or water, that is consumed by and/or administered to a human.

Advantageously, the compositions, methods and equipment described herein can reduce the capital and labor costs of producing microorganisms and their metabolites on a large scale. Furthermore, the subject invention provides a cultivation method that substantially increases the yield of microbial products per unit of nutrient medium, simplifies production, facilitates portability, and minimizes waste.

### DETAILED DESCRIPTION OF THE INVENTION

The subject invention provides microbe-based compositions, as well as methods for producing them. Methods are also provided for using these compositions in a variety of applications, including animal feed, bioremediation, mining and/or bioleaching, agriculture, oil and gas recovery, human health, and many others.

Specifically, the subject microbe-based compositions are yeast fermentation products obtained during production of biosurfactants, such as, e.g., sophorolipids (SLP). Through submerged cultivation of a biosurfactant-producing microorganism, biosurfactants are excreted into the fermentation broth and then harvested for further processing and/or purification. What remains in the culture is a product with several different utilities. Advantageously, when produced according to the subject invention, recycling of these leftover culture products can reduce waste and increase the profitability of biosurfactant production particularly for large-scale, commercial operations.

In certain embodiments, the subject invention provides a yeast fermentation product comprising supernatant and, optionally, yeast cell biomass, resulting from cultivation of a yeast microbe. Preferably, the yeast is a biosurfactant-producing yeast that has been cultivated for the purpose of producing a biosurfactant. Even more preferably, the yeast is Starmerella bombicola, which is capable of producing sophorolipid (SLP) biosurfactants in high concentrations.

### Selected Definitions

As used herein, a “biofilm” is a complex aggregate of microorganisms, such as bacteria, wherein the cells adhere to each other and/or to a surface via an extracellular polysaccharide matrix. The cells in biofilms are physiologically distinct from planktonic cells of the same organism, which are single cells that can float or swim in liquid medium.

As used herein, the term “control” used in reference to a pest or other undesirable organism extends to the act of killing, disabling or immobilizing the pest or other organism, or otherwise rendering the pest or other organism substantially incapable of causing harm.

As used herein, “harvested” in the context of microbial fermentation refers to removing some or all of a microbe-based composition from a growth vessel.

As used herein, “intermediate bulk container,” “IBC” or “pallet tank” refers to a reusable industrial container designed for transporting and storing bulk substances, including, e.g., chemicals (including hazardous materials), food ingredients (e.g., syrups, liquids, granulated and powdered ingredients), solvents, detergents, adhesives, water and pharmaceuticals. Typically, IBCs are stackable and mounted on a pallet designed to be moved using a forklift or a pallet jack. Thus, IBCs are designed to enable portability.

As used herein, an “isolated” or “purified” nucleic acid molecule, polynucleotide, polypeptide, protein, organic compound such as a small molecule (e.g., those described below), or other compound is substantially free of other compounds, such as cellular material, with which it is associated in nature. For example, a purified or isolated polynucleotide (ribonucleic acid (RNA) or deoxyribonucleic acid (DNA)) is free of the genes or sequences that flank it in its naturally-occurring state. A purified or isolated polypeptide is free of the amino acids or sequences that flank it in its naturally-occurring state. A purified or isolated microbial strain is removed from the environment in which it exists in nature. Thus, the isolated strain may exist as, for example, a biologically pure culture, or as spores (or other forms of the strain) in association with a carrier.

In certain embodiments, purified compounds are at least 60% by weight the compound of interest. Preferably, the preparation is at least 75%, more preferably at least 90%, and most preferably at least 99%, by weight the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis.

A “metabolite” refers to any substance produced by metabolism (e.g., a growth by-product) or a substance necessary for taking part in a particular metabolic process. A metabolite can be an organic compound that is a starting material, an intermediate in, or an end product of metabolism. Examples of metabolites can include, but are not limited to, enzymes, toxins, acids, solvents, alcohols, proteins, carbohydrates, vitamins, minerals, microelements, amino acids, polymers, and surfactants.

As used herein, reference to a “microbe-based composition” means a composition that comprises components that were produced as the result of the growth of microorganisms or other cell cultures. Thus, the microbe-based composition may comprise the microbes themselves and/or by-products of microbial growth. The microbes may be in a vegetative state, in spore form, in mycelial form, in any other form of microbial propagule, or a mixture of these. The microbes may be planktonic or in a biofilm form, or a mixture of both. The by-products of growth may be, for example, metabolites (e.g., biosurfactants), cell membrane components, expressed proteins, and/or other cellular components. The microbes may be intact or lysed. The cells may be totally absent, or present at, for example, a concentration of at least 1×10⁴, 1×10⁵, 1×10⁶, 1×10⁷, 1×10⁸, 1×10⁹, 1 ×10¹, 1×10¹¹, 1×10¹², 1×10¹³ or more CFU/ml of the composition.

The subject invention further provides “microbe-based products,” which are products that are to be applied in practice to achieve a desired result. The microbe-based product can be simply the microbe-based composition harvested from the microbe cultivation process. Alternatively, the microbe-based product may comprise further ingredients that have been added. These additional ingredients can include, for example, stabilizers, buffers, carriers (e.g., water or salt solutions), added nutrients to support further microbial growth, non-nutrient growth enhancers and/or agents that facilitate tracking of the microbes and/or the composition in the environment to which it is applied. The microbe-based product may also comprise mixtures of microbe-based compositions. The microbe-based product may also comprise one or more components of a microbe-based composition that have been processed in some way such as, but not limited to, filtering, centrifugation, lysing, drying, purification and the like.

As used herein, “surfactant” refers to a compound that lowers the surface tension (or interfacial tension) between two liquids or between a liquid and a solid. Surfactants act as detergents, wetting agents, emulsifiers, foaming agents, and dispersants. A “biosurfactant” is a surfactant produced by a living organism.

The transitional term “comprising,” which is synonymous with “including,” or “containing,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention. Use of the term “comprising” contemplates embodiments “consisting” and “consisting essentially” of the recited component(s).

Unless specifically stated or obvious from context, as used herein, the term “or” is understood to be inclusive. Unless specifically stated or obvious from context, as used herein, the terms “a,” “and” and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

All references cited herein are hereby incorporated by reference in their entirety.

### Yeast Fermentation Product

The subject invention provides microbe-based compositions, as well as methods for producing them. Methods are also provided for using these composition in a variety of applications, including animal feed, bioremediation, mining and/or bioleaching, agriculture, oil and gas recovery, human health, and many others.

Specifically, the subject microbe-based compositions are yeast fermentation products obtained during production of biosurfactants, such as, e.g., sophorolipids (SLP). Through submerged cultivation of a biosurfactant-producing microorganism, biosurfactants are excreted into the fermentation broth and then harvested for further processing and/or purification. What remains in the culture is a product with several different utilities. Advantageously, when produced according to the subject invention, recycling of these leftover culture products can reduce waste and increase the profitability of biosurfactant production particularly for large-scale, commercial operations.

Biosurfactants have excellent surface and interfacial tension reduction properties, as well as other beneficial biochemical properties, which can be useful in applications such as large scale industrial uses. Safe, effective microbial biosurfactants reduce the surface and interfacial tensions between the molecules of liquids, solids, and gases. Furthermore, biosurfactants accumulate at interfaces, thus reducing interfacial tension and leading to the formation of aggregated micellar structures in solution. Thus, advantageously, the ability of biosurfactants to form pores and destabilize biological membranes permits their use as, for example, antimicrobial and hemolytic agents.

In certain embodiments, the subject invention provides a yeast fermentation product comprising supernatant and, optionally, yeast cell biomass, resulting from cultivation of a yeast microbe.

The microorganisms useful according to the subject invention may be natural, or genetically modified microorganisms. For example, the microorganisms may be transformed with specific genes to exhibit specific characteristics. The microorganisms may also be mutants of a desired strain. As used herein, “mutant” means a strain, genetic variant or subtype of a reference microorganism, wherein the mutant has one or more genetic variations (e.g., a point mutation, missense mutation, nonsense mutation, deletion, duplication, frameshift mutation or repeat expansion) as compared to the reference microorganism. Procedures for making mutants are well known in the microbiological art. For example, UV mutagenesis and nitrosoguanidine are used extensively toward this end.

In one embodiment, the microorganisms are yeasts and/or fungi. Yeast and fungus species suitable for use according to the current invention, include Acaulospora, Acremonium chrysogenum, Aspergillus, Aureobasidium (e.g., A. pullulans), Blakeslea, Candida (e.g., C. albicans, C. apicola, C. batistae, C. bombicola, C. floricola, C. kuoi, C. riodocensis, C. nodaensis, C. stellate), Cryptococcus, Debaryomyces (e.g., D. hansenii), Entomophthora, Hanseniaspora (e.g., H. uvarum), Hansenula, Issatchenkia, Kluyveromyces (e.g., K. phaffii), Lentinula spp. (e.g., L. edodes), Meyerozyma (e.g., M. guilliermondii), Monascus purpureus, Mortierella, Mucor (e.g., M. piriformis), Penicillium, Phythium, Phycomyces, Pichia (e.g., P. anomala, P. guilliermondii, P. occidentalis, P. kudriavzevii), Pleurotus (e.g., P. ostreatus P. ostreatus, P. sajorcaju, P. cystidiosus, P. cornucopiae, P. pulmonarius, P. tuberregium, P. citrinopileatus and P. flabellatus), Pseudozyma (e.g., P. aphidis), Rhizopus, Rhodotorula (e.g., R. bogoriensis); Saccharomyces (e.g., S. cerevisiae, S. boulardii, S. torula), Starmerella (e.g., S. bombicola), Torulopsis, Thraustochytrium, Trichoderma (e.g., T. reesei, T. harzianum, T viridae), Ustilago (e.g., U. maydis), Wickerhamiella (e.g., W. domericqiae), Wickerhamomyces (e.g., W. anomalus), Williopsis (e.g., W. mrakii), Zygosaccharomyces (e.g., Z. bacilii), and others.

Preferably, the yeast is a biosurfactant-producing yeast that has been cultivated for the purpose of producing a biosurfactant. Even more preferably, the yeast is Starmerella bombicola, which is capable of producing sophorolipid (SLP) biosurfactants in high concentrations.

Sophorolipids are glycolipid biosurfactants that comprise a disaccharide sophorose linked to long chain hydroxy fatty acids. They can comprise a partially acetylated 2-O-β-D-glucopyranosyl-D-glucopyranose unit attached β-glycosidically to 17-L-hydroxyoctadecanoic or 17-L-hydroxy-Δ9-octadecenoic acid. The hydroxy fatty acid is generally 16 or 18 carbon atoms, and may contain one or more unsaturated bonds. Furthermore, the sophorose residue can be acetylated on the 6- and/or 6′-position(s). The fatty acid carboxyl group can be free (acidic or linear form) or internally esterified at the 4″-position (lactonic form). S. bombicola produces a specific enzyme, called S. bombicola lactone esterase, which catalyzes the esterification of linear SLP to produce lactonic SLP.

The system can also utilize one or more strains of yeast related thereto, such as, e.g., Candida apicola, Candida batistae, Candida floricola, Candida riodocensis, Candida stellate, Candida kuoi, as well as any other glycolipid-producing strains of the Candida and/or Starmerella clades. In a specific embodiment, the yeast strain is ATCC 22214 and mutants thereof.

Other microbial strains including strains capable of accumulating significant amounts of, for example, glycolipid-biosurfactants (e.g., rhamnolipids, mannosylerythritol lipids and/or trehalose lipids), lipopeptide biosurfactants (e.g., surfactin, iturin, fengycin and/or lichenysin), mannoprotein, beta-glucan, enzymes, acids, biopolymers, proteins, vitamins, minerals, and/or solvents, can be used in accordance with the subject invention.

In one embodiment, the supernatant of the yeast fermentation product comprises yeast growth by-products, such as, e.g., excreted metabolites and/or cell wall components. In some embodiments, the growth by-product is a residual biosurfactant.

In certain embodiments, the yeast fermentation products according to the subject invention can be superior to, for example, purified microbial metabolites alone, due to, for example, the advantageous properties of the yeast cell walls. These properties include high concentrations of mannoprotein, as well as the biopolymer beta-glucan as a part of a yeast cell wall's outer surface. These compounds can serve as, for example, effective emulsifiers. Additionally, the yeast fermentation product further can comprise residual biosurfactants in the culture, as well as other metabolites and/or cellular components, such as solvents, acids, vitamins, minerals, enzymes and proteins. Thus, the yeast fermentation products can, among many other uses, act as biosurfactants and can have surface/interfacial tension-reducing properties.

### Methods of Cultivation

The subject invention further provides methods for cultivation of microorganisms and production of microbial metabolites and/or other by-products of microbial growth. In one embodiment, the subject invention provides materials and methods for the production of biomass (e.g., viable cellular material), extracellular metabolites (e.g. small molecules and excreted proteins), residual nutrients and/or intracellular components (e.g. enzymes and other proteins).

In specific embodiments, the methods can be used for producing the subject yeast fermentation product, wherein the methods comprise cultivating a yeast in culture medium using a submerged fermentation system; allowing the yeast to produce a biosurfactant into the medium; and harvesting the biosurfactant to leave behind supernatant and yeast cell biomass in the fermentation system. The supernatant and yeast cell biomass that are left behind comprise the yeast fermentation product.

In certain embodiments, the methods of cultivation comprise adding a culture medium comprising water and nutrient components to a fermentation system using, for example, a peristaltic pump; inoculating the system with a viable microorganism; and optionally, adding an antimicrobial agent to the culture medium. The antimicrobial agent can be, for example, an antibiotic or a biosurfactant (e.g., SLP).

In one embodiment, the method comprises inoculating a fermentation reactor comprising a liquid growth medium with a sophorolipid-producing yeast to produce a yeast culture; and cultivating the yeast culture under conditions favorable for production of SLP.

The microbe growth vessel used according to the subject invention can be any fermenter or cultivation reactor for industrial use. In one embodiment, the vessel may have functional controls/sensors or may be connected to functional controls/sensors to measure important factors in the cultivation process, such as pH, oxygen, pressure, temperature, agitator shaft power, humidity, viscosity and/or microbial density and/or metabolite concentration.

In a further embodiment, the vessel may also be able to monitor the growth of microorganisms inside the vessel (e.g., measurement of cell number and growth phases). Alternatively, samples may be taken from the vessel for enumeration, purity measurements, SLP concentration, and/or visible oil level monitoring. For example, in one embodiment, sampling can occur every 24 hours.

The microbial inoculant according to the subject methods preferably comprises cells and/or propagules of the desired microorganism, which can be prepared using any known fermentation method. The inoculant can be pre-mixed with water and/or a liquid growth medium, if desired.

In certain embodiments, the cultivation method utilizes submerged fermentation in a liquid growth medium. The culture medium can be formulated for enhanced biosurfactant production.

In one embodiment, the liquid growth medium comprises a carbon source. The carbon source can be a carbohydrate, such as glucose, dextrose, sucrose, lactose, fructose, trehalose, mannose, mannitol, and/or maltose; organic acids such as acetic acid, fumaric acid, citric acid, propionic acid, malic acid, malonic acid, and/or pyruvic acid; alcohols such as ethanol, propanol, butanol, pentanol, hexanol, isobutanol, and/or glycerol; fats and oils such as canola oil, soybean oil, rice bran oil, olive oil, corn oil, sunflower oil, sesame oil, and/or linseed oil; powdered molasses, etc. These carbon sources may be used independently or in a combination of two or more. In preferred embodiments, a hydrophilic carbon source, e.g., glucose, and a hydrophobic carbon source, e.g., oil or fatty acids, are used.

In one embodiment, the liquid growth medium comprises a nitrogen source. The nitrogen source can be, for example, yeast extract, potassium nitrate, ammonium nitrate, ammonium sulfate, ammonium phosphate, ammonia, urea, and/or ammonium chloride. These nitrogen sources may be used independently or in a combination of two or more.

In one embodiment, one or more inorganic salts may also be included in the liquid growth medium. Inorganic salts can include, for example, potassium dihydrogen phosphate, monopotassium phosphate, dipotassium hydrogen phosphate, disodium hydrogen phosphate, potassium chloride, magnesium sulfate, magnesium chloride, iron sulfate, iron chloride, manganese sulfate, manganese chloride, zinc sulfate, lead chloride, copper sulfate, calcium chloride, calcium carbonate, calcium nitrate, magnesium sulfate, sodium phosphate, sodium chloride, and/or sodium carbonate. These inorganic salts may be used independently or in a combination of two or more.

In one embodiment, growth factors and trace nutrients for microorganisms are included in the medium. This is particularly preferred when growing microbes that are incapable of producing all of the vitamins they require. Inorganic nutrients, including trace elements such as iron, zinc, copper, manganese, molybdenum and/or cobalt may also be included in the medium. Furthermore, sources of vitamins, essential amino acids, proteins and microelements can be included, for example, corn flour, peptone, yeast extract, potato extract, beef extract, soybean extract, banana peel extract, and the like, or in purified forms. Amino acids such as, for example, those useful for biosynthesis of proteins, can also be included.

The method of cultivation can further provide oxygenation to the growing culture. One embodiment utilizes slow motion of air to remove low-oxygen containing air and introduce oxygenated air. The oxygenated air may be ambient air supplemented daily through mechanisms including impellers for mechanical agitation of the liquid, and air spargers for supplying bubbles of gas to the liquid for dissolution of oxygen into the liquid.

The cultivation processes of the subject invention can be anaerobic, aerobic, or a combination thereof. Preferably, the process is aerobic, keeping the dissolved oxygen (DO) concentration above 10 or 15% of saturation during fermentation, but within 20% in some embodiments, or within 30% in some embodiments.

In certain embodiments, DO levels are maintained at about 25% to about 75%, about 30% to about 70%, about 35% to about 65%, about 40% to about 60%, or about 50% of air saturation.

In some embodiments, the method for cultivation may further comprise adding additional acids and/or antimicrobials in the liquid medium before and/or during the cultivation process. Antimicrobial agents or antibiotics (e.g., streptomycin, oxytetracycline) are used for protecting the culture against contamination. In some embodiments, however, the metabolites produced by the yeast culture provide sufficient antimicrobial effects to prevent contamination of the culture.

In one embodiment, prior to inoculation of the system, the fermentation medium, air, and equipment can be sterilized or disinfected.

In one embodiment, prior to inoculation, the components of the liquid culture medium can optionally be sterilized. In one embodiment, sterilization of the liquid growth medium can be achieved by placing the components of the liquid culture medium in water at a temperature of about 85-100° C. In one embodiment, sterilization can be achieved by dissolving the components in 1 to 3% hydrogen peroxide in a ratio of 1:3 (w/v). In another embodiment, all nutritional and other medium components can be autoclaved prior to fermentation. In other embodiments, the medium may be pasteurized or, optionally, no heat at all added, where the use of pH and/or low water activity may be exploited to control unwanted microbial growth.

In a specific embodiment, the water used in the culture medium is UV sterilized using an in-line UV water sterilizer and filtered using, for example, a 0.1-micron water filter.

In one embodiment, the equipment used for cultivation is sterile. The cultivation equipment such as the reactor/vessel may be separated from, but connected to, a sterilizing unit, e.g., an autoclave. The cultivation equipment may also have a sterilizing unit that sterilizes in situ before starting the inoculation. Gaskets, openings, tubing and other equipment parts can be sprayed with, for example, isopropyl alcohol. Air can be sterilized by methods know in the art. For example, the ambient air can pass through at least one filter before being introduced into the vessel.

In one embodiment, the internal surfaces of the reactor (including, e.g., tanks, ports, spargers and mixing systems) can first be washed with a commercial disinfectant; then fogged (or sprayed with a highly dispersed spray system) with 2% to 4% hydrogen peroxide, preferably 3% hydrogen peroxide; and finally steamed with a portable steamer at a temperature of about 105° C. to about 110° C., or greater.

In yet another embodiment, the fermentation vessel can be sterilized with a hydrogen peroxide solution (e.g., from 2.0% to 4.0% hydrogen peroxide; this can be performed before or after a hot water rinse at, e.g., 80-90° C.) to prevent contamination. In addition, or in the alternative, the tank can be washed with a commercial disinfectant, a bleach solution and/or a hot water or steam rinse. The system can come with concentrated forms of the bleach and hydrogen peroxide, which can later be diluted at the fermentation site before use. For example, the hydrogen peroxide can be provided in concentrated form and be diluted to formulate 2.0% to 4.0% hydrogen peroxide (by weight or volume) for pre-rinse decontamination.

In other embodiments, the cultivation system may be self-sterilizing, meaning the organism being cultivated is capable of preventing contamination from other organisms due to production of antimicrobial growth by-products (e.g., biosurfactants).

The pH of the culture should be suitable for the microorganism of interest. In some embodiments, the pH is about 2.0 to about 5.0, about 3.0 to about 4.0, about 3.25 to about 3.75, or about 3.5. In preferred embodiments, the p11 is about 3.5 +/−0.05. Buffers, and pH regulators, such as carbonates and phosphates, may be used to stabilize pH near a preferred value. In certain embodiments, a base solution is used to adjust the pH of the culture to a favorable level, for example, a 15% to 30%, or a 20% to 25% NaOH. The base solution can be included in the growth medium and/or it can be fed into the fermentation reactor during cultivation to adjust the pH as needed.

pH control can also be used for preventing contamination of the culture. For example, cultivation can be initiated at low pH that is suitable for yeast growth (e.g., 3.0-3.5), and then increased after yeast accumulation (e.g., to 4.5-5.0) and stabilized for the remainder of fermentation. The fermentation can also start at a higher first pH (e.g., a pH of 4.0 to 4.5) and later change to a second, lower pH (e.g., a pH of 3.2-3.5) for the remainder of the process to help avoid contamination as well as to produce other desirable results.

In some embodiments, pH is adjusted from a first pH to a second pH after a desired accumulation of biomass is achieved, for example, from 0 hours to 200 hours after the start of fermentation, more specifically from 12 to 120 hours after, more specifically from 24 to 72 hours after. When metal ions are present in high concentrations, use of a chelating agent in the liquid medium may be necessary.

According to the subject methods, the microorganisms can be incubated in the fermentation system for a time period sufficient to achieve a desired effect, e.g., production of a desired amount of cell biomass or a desired amount of one or more microbial growth by-products. The microbial growth by-product(s) produced by microorganisms may be retained in the microorganisms and/or secreted into the growth medium. The biomass content may be, for example from 5 g/l to 180 g/l or more, or from 10 g/l to 150 g/l.

The microbes can be grown in planktonic form or as biofilm. In the case of biofilm, the vessel may have within it a substrate upon which the microbes can be grown in a biofilm state. The system may also have, for example, the capacity to apply stimuli (such as shear stress) that encourages and/or improves the biofilm growth characteristics.

In one embodiment, the method of cultivation is carried out at about 5° to about 100° C., about 15° to about 60° C., about 20° to about 45° C., about 22° to about 30° C., or about 24° to about 28° C. In one embodiment, the cultivation may be carried out continuously at a constant temperature. In another embodiment, the cultivation may be subject to changing temperatures.

In one embodiment, the moisture level of the culture medium should be suitable for the microorganism of interest. In a further embodiment, the moisture level may range from 20% to 90%, preferably, from 30 to 80%, more preferably, from 40 to 60%.

In one embodiment, total fermentation times can range from 12 hours to 14 days, more preferably from 1 day to 10 days, even more preferably from 2 to 7 days, or 3 to 5 days.

In one embodiment, a single type of microbe is grown in a vessel. In alternative embodiments, multiple microbes, which can be grown together without deleterious effects on growth or the resulting product, can be grown in a single vessel. There may be, for example, 2 to 3 or more different microbes grown in a single vessel at the same time.

In an exemplary embodiment, cultivation of the yeast occurs at 25-28° C. for 1 to 10 days. After the submerged fermentation cycle is complete, e.g., when glucose concentration and/or oil concentration in the fermentation medium reaches 0%, the entire yeast culture is left to sit with minimal or no disturbance, either in the fermentation reactor, or after being collected into a separate, first collection container. A layer of SLP derivatives will settle at the bottom of the sitting culture, comprising about 10-15% SLP, or about 4-5 g/L. Advantageously, once the SLP layer is harvested from the culture, about 1-4 g/L of SLP can still remain in the yeast fermentation product, as well as other advantageous yeast growth by-products and cellular components.

In one embodiment, the subject invention provides methods of producing a microbial metabolite by cultivating a microorganism under conditions appropriate for growth and production of the metabolite; harvesting the metabolite; and, optionally, purifying the metabolite. In a specific embodiment, the metabolite is a biosurfactant. The metabolite may also be, for example, ethanol, lactic acid, beta-glucan, proteins, amino acids, peptides, metabolic intermediates, polyunsaturated fatty acids, and lipids. The metabolite content produced by the method can be, for example, at least 10%, 20%, 30%, 40%, 50%, 60%, 70%, 80%, or 90%.

In some specific embodiments, the methods can be used for producing a biosurfactant product, wherein, after harvesting the SLP sediment layer from the culture medium, the method comprises processing and/or purifying the SLP, if desired. In one embodiment, the biosurfactants are left in a crude and/or unpurified form. Crude form biosurfactants can take the form of a mixture comprising the SLP sediment, with some residual amounts of fermentation broth. This crude form SLP can comprise from about 0.001% to about 99%, about 25% to about 75%, about 30% to about 70%, about 35% to about 65%, about 40% to about 60%, about 45% to about 55%, or about 50% pure SLP.

The microbial growth by-product produced by microorganisms of interest may be retained in the microorganisms or secreted into the growth medium. In another embodiment, the method for producing microbial growth by-product may further comprise steps of concentrating and purifying the microbial growth by-product of interest. In a further embodiment, the medium may contain compounds that stabilize the activity of microbial growth by-product.

In one embodiment, rather than using liquid medium, the microorganisms can be grown on a solid or semi-solid substrate, such as, for example, corn, wheat, soybean, chickpeas, beans, oatmeal, pasta, rice, and/or flours or meals of any of these or other similar substances. This resulting culture and substrate can be blended and dissolved in water or another solvent, and then, for example, centrifuged to separate the components and extract the biosurfactants therefrom.

The subject methods for cultivation of microorganisms and production of the microbial by-products can be performed in a batch, quasi-continuous, or continuous processes.

In one embodiment, all of the microbial cultivation composition is removed upon the completion of the cultivation (e.g., upon, for example, achieving a desired cell density, or density of a specified metabolite). In this batch procedure, an entirely new batch is initiated upon harvesting of the first batch.

In another embodiment, only a portion of the fermentation product is removed at any one time. In this embodiment, biomass with viable cells remains in the vessel as an inoculant for a new cultivation batch. The composition that is removed can be a microbe-free medium or contain cells, spores, mycelia, conidia or other microbial propagules. In this manner, a quasi-continuous system is created.

In one embodiment, the subject invention further provides customizations to the materials and methods according to the local needs. For example, the method for cultivation of microorganisms may be used to grow those microorganisms located in the local soil or at a specific oil well or site of pollution. In specific embodiments, local soils may be used as the solid substrates in the cultivation method for providing a native growth environment. Advantageously, these microorganisms can be beneficial and more adaptable to local needs.

The cultivation method according to the subject invention not only substantially increases the yield of microbial products per unit of nutrient medium but also improves the simplicity of the production operation. Furthermore, the cultivation process can eliminate or reduce the need to concentrate microorganisms after finalizing fermentation.

Advantageously, the method does not require complicated equipment or high energy consumption, and thus reduces the capital and labor costs of producing microorganisms and their metabolites on a large scale. Similarly, the microbial metabolites can also be produced at large quantities at the site of need.

### Preparation and Use of Yeast Fermentation Products

The subject invention provides microbe-based products and methods for using these products in a variety of applications, including animal feed, bioremediation, mining and/or bioleaching, agriculture, oil and gas recovery, human health, and many others.

In certain embodiments, the subject microbe-based compositions are yeast fermentation products obtained during production of biosurfactants, such as, e.g., sophorolipids (SLP). In certain specific embodiments, the yeast fermentation comprises supernatant and, optionally, yeast cell biomass, resulting from cultivation of a yeast microbe. The supernatant can comprise residual microbial metabolites, including residual biosurfactants, as well as any remaining nutrients.

Preferably, the yeast is a biosurfactant-producing yeast that has been cultivated for the purpose of producing a biosurfactant. Even more preferably, in certain embodiments, the yeast is Starmerella bombicola, which is capable of producing sophorolipid (SLP) biosurfactants in high concentrations.

The characteristics and formulation, e.g., nutrients, carriers, additives, and/or yeast cell concentration, of the yeast fermentation product can be optimized depending upon the desired application.

The product of fermentation may be used directly without extraction or purification. If desired, extraction and purification can be easily achieved using standard extraction and/or purification methods or techniques described in the literature.

The product can be formulated as, for example, a liquid suspension, an emulsion, a freeze- or spray-dried powder, pellets, granules, gels, or other forms depending on mode of application. In certain preferred embodiments, however, the composition is utilized in liquid form with little to no processing after harvesting from the vessel in which it was cultivated.

The microorganisms in the composition may be in an active or inactive form and/or in the form of vegetative cells, spores, mycelia, conidia and/or any form of microbial propagule. The composition may or may not comprise the growth medium in which the microbes were grown. The composition may also be in a dried form or a liquid form.

The microbe-based products may be used without further stabilization, preservation, and storage. Advantageously, direct usage of these microbe-based products preserves a high viability of the microorganisms, reduces the possibility of contamination from foreign agents and undesirable microorganisms, and maintains the activity of the by-products of microbial growth.

The microbes and/or medium (e.g., broth or, in certain cases, solid substrate) resulting from the microbial growth can be removed from the growth vessel and transferred via, for example, piping for immediate use.

In other embodiments, the microbe-based product (microbes, medium, or microbes and medium) can be placed in containers of appropriate size, taking into consideration, for example, the intended use, the contemplated method of application, the size of the fermentation vessel, and any mode of transportation from microbe growth facility to the location of use. Thus, the containers into which the microbe-based composition is placed may be, for example, from 1 gallon to 1,000 gallons or more. In other embodiments the containers are 2 gallons, 5 gallons, 25 gallons, or larger.

Advantageously, in accordance with the subject invention, the microbe-based product may comprise broth in which the microbes were grown. The product may be, for example, at least, by weight, 1%, 5%, 10%, 25%, 50%, 75%, or 100% broth. The amount of biomass in the product, by weight, may be, for example, anywhere from 0% to 100% inclusive of all percentages therebetween.

Upon harvesting, for example, the yeast fermentation product, from the growth vessels, further components can be added as the harvested product is placed into containers and/or piped (or otherwise transported for use). The additives can be, for example, buffers, carriers, other microbe-based compositions produced at the same or different facility, viscosity modifiers, preservatives, nutrients for microbe growth, tracking agents, pH adjusting agents, stabilizers, ultra-violet light resistant agents, emulsifying agents, lubricants, solvents, solubility controlling agents, biocides, other microbes and other ingredients specific for an intended use.

In one embodiment, the product may comprise buffering agents including organic and amino acids or their salts. Suitable buffers include citrate, gluconate, tartarate, malate, acetate, lactate, oxalate, aspartate, malonate, glucoheptonate, pyruvate, galactarate, glucarate, tartronate, glutamate, glycine, lysine, glutamine, methionine, cysteine, arginine and a mixture thereof. Phosphoric and phosphorous acids or their salts may also be used. Synthetic buffers are suitable to be used but it is preferable to use natural buffers such as organic and amino acids or their salts listed above.

In a further embodiment, pH adjusting agents include potassium hydroxide, ammonium hydroxide, potassium carbonate or bicarbonate, hydrochloric acid, nitric acid, sulfuric acid or a mixture.

In one embodiment, an aqueous preparation of a salt, such as sodium bicarbonate or carbonate, sodium sulfate, sodium phosphate, sodium biphosphate, can be included in the formulation.

In one embodiment, additional components can be included to increase the efficacy of the treatment products, such as chelating agents and adherents.

Optionally, the product can be stored prior to use. The storage time is preferably short. Thus, the storage time may be less than 60 days, 45 days, 30 days, 20 days, 15 days, 10 days, 7 days, 5 days, 3 days, 2 days, 1 day, or 12 hours. In a preferred embodiment, if live cells are present in the product, the product is stored at a cool temperature such as, for example, less than 20° C., 15° C., 10° C., or 5° C.

In certain embodiments, methods for using the subject yeast fermentation product are provided, wherein the methods comprise applying a yeast fermentation product according to embodiments of the subject invention to a target application site. The product can be harvested from the fermentation system and applied directly to the target site. In other embodiments, the product is stored and/or transported after harvesting and prior to application to the target site.

As used herein, “applying” a composition or product to a target or site, or “treating” a target or site, refers to contacting a composition or product with a target or site such that the composition or product can have an effect on that target or site. The effect can be due to, for example, microbial growth and/or the action of a metabolite, enzyme, biosurfactant or other growth by-product. Application or treatment can include spraying, sprinkling, pouring, injecting, mixing, dunking, spreading, painting, or any other conceivable means of contacting a composition with a target site.

In one exemplary embodiment, the yeast fermentation product can be used as a nutritional supplement and/or digestive aide for an animal, wherein the target application site is an animal's feed and/or drinking water. The product can be formulated as and/or mixed with feed and/or water.

In one exemplary embodiment, the yeast fermentation product can be used as a bioremediation agent, wherein the target application site is a site, such as water or soil, that has been contaminated with, for example, oil from an oil spill, or a toxic substance, such as arsenic.

In one exemplary embodiment, the yeast fermentation product can be used as a mining and/or bioleaching agent, wherein the target application site is ore and/or coal that contains one or more valuable, minable elements, such as a precious metal or rare earth metal.

In one exemplary embodiment, the yeast fermentation product can be used for enhanced agricultural production, wherein the target application site is soil, a plant and/or a seed. In one exemplary embodiment, the yeast fermentation product can be used for enhanced oil recovery (EOR), wherein the target application site is an oil and/or gas well, a subterranean formation, and/or any piece of equipment associated with oil and/or gas recovery, transport, refining, and processing.

In one exemplary embodiment, the yeast fermentation product can be used as an adjuvant composition and/or as a digestive aide in humans, wherein the target site is a pharmaceutical, a supplement, a nutrient and/or water, that is consumed by and/or administered to a human.

### EXAMPLES

A greater understanding of the present invention and of its many advantages may be had from the following examples, given by way of illustration. The following examples are illustrative of some of the methods, applications, embodiments and variants of the present invention. They are not to be considered as limiting the invention. Numerous changes and modifications can be made with respect to the invention.

### Example 1

### Fermentation Reactor System

In certain embodiments, the subject invention utilizes fermentation systems for producing microbe-based composition. The system can include all of the materials necessary for the fermentation (or cultivation) process, including, for example, equipment, sterilization supplies, and culture medium components, although it is expected that freshwater could be supplied from a local source and sterilized according to the subject methods.

One example of a system comprises one high volume, vertical parallelepiped tank. The tank can be any fermenter or cultivation reactor for industrial use. The tank may be made of, for example, glass, polymers, metals, metal alloys, and combinations thereof. Preferably, the tank is made of metal, for example, stainless steel. In one embodiment, the tank is a modified stainless steel intermediate bulk container (“IBC”).

The system can be scaled depending on the intended use. For example, the system can be as small as 50 gallons or even smaller, or the system can be scaled to produce 20,000 gallons or more of product.

The tank can range in size from a few gallons to tens of thousands of gallons. The tank may be, for example, from 5 liters to 5,000 liters or more. Typically, the tank will be from 10 to 4,000 liters, and preferably from 100 to 2,500 liters.

In an exemplary embodiment, the tank has a volume of 550 gallons (about 2,082 liters) and can measure 5 by 5 feet in length and width, and 6 feet 4 inches in height.

The system can be equipped with one or more of: pH stabilization capabilities, temperature controls, an automated system for running a steam sterilization cycle; an impeller, or other form of mixing device; an external circulation system; and an aeration system or an air compressor.

In one embodiment, the external circulation system comprises two highly efficient external loops comprising inline heat exchangers. In one embodiment, the heat exchangers are shell-and-tube heat exchangers. Each loop is fitted with its own circulation pump.

The two pumps transport liquid from the bottom of the tank at, for example, 250 to 400 gallons per minute, through the heat exchangers, and back into the top of the tank. Advantageously, the high velocity at which the culture is pumped through the loops helps prevent cells from caking on the inner surfaces thereof.

The loops can be attached to a water source and, optionally, a chiller, whereby the water is pumped with a flow rate of about 10 to 15 gallons per minute around the culture passing inside the heat exchangers, thus increasing or decreasing temperature as desired. In one embodiment, the water controls the temperature of the culture without ever contacting the culture.

The reactor system can further comprise an aeration system capable of providing filtered air to the culture. The aeration system can, optionally, have an air filter for preventing contamination of the culture. The aeration system can function to keep the air level over the culture, the dissolved oxygen (DO), and the pressure inside the tank, at desired (e.g., constant) levels.

In certain embodiments, the unit can be equipped with a unique sparging system, through which the aeration system supplies air. Preferably, the sparging system comprises stainless steel injectors that produce microbubbles. In an exemplary embodiment, the spargers can comprise from 4 to 10 aerators, comprising stainless steel microporous pipes (e.g., having tens or hundreds of holes 1 micron or less in size), which are connected to an air supply. The unique microporous design allows for proper dispersal of oxygen throughout the culture, while preventing contaminating microbes from entering the culture through the air supply.

In some embodiments, the reactor system is controlled by a programmable logic controller (PLC). In certain embodiments, the PLC has a touch screen and/or an automated interface. The PLC can be used to start and stop the reactor system, and to monitor and adjust, for example, temperature, DO, and pH, throughout fermentation.

The reactor system can be equipped with probes for monitoring fermentation parameters, such as, e.g., pH, temperature and DO levels. The probes can be connected to a computer system, e.g., the PLC, which can automatically adjust fermentation parameters based on readings from the probes.

In certain embodiments, the DO is adjusted continuously as the microorganisms of the culture consume oxygen and reproduce. For example, the oxygen input can be increased steadily as the microorganisms grow, in order to keep the DO constant at about 30% (of saturation).

The reactor system can also be equipped with a system for running a steam sterilization cycle before and/or after running the reactor system. In certain embodiments, the steam sterilization system is automated.

The reactor system can comprise an off-gas system to release air. De-foaming measures can also be employed to suppress foam production, such as mechanical anti-foam apparatuses or chemical or biochemical additives.

In one embodiment, the system is provided with an inoculum of viable microbes. Preferably, the microbes are biochemical-producing microbes, capable of accumulating, for example, biosurfactants, enzymes, solvents, biopolymers, acids, and/or other useful metabolites. In particularly preferred embodiments, the microorganisms are biochemical-producing yeast, fungi, and/or bacteria.

In one embodiment, the system is provided with a culture medium. The medium can include nutrient sources, for example, a protein source, a carbon source, a lipid source, a nitrogen source, and/or a source of other nutrients, such as micronutrients. Each of these nutrient sources can be provided in an individual package that can be added to the reactor at appropriate times during the fermentation process. Each of the packages can include several sub-packages that can be added at specific points (e.g, when yeast, pH, and/or nutrient levels go above or below a specific concentration) or times (e.g., after 10 hours, 20 hours, 30 hours, 40 hours, etc.) during the fermentation process.

### Example 2

### Production Of Sophorolipids And The Yeast Fermentation Product

A system as described in Example 1 can be used for production of sophorolipids and the yeast fermentation product of the subject invention. The reactor comprises about 150 gallons of water, into which a medium comprising dextrose (25 to 150 g/L), yeast extract (1 to 10 g/L), canola oil (25 ml/L to 110 ml/L) and urea (0.5 to 5 g/L) is added.

The reactor comprises a mixing apparatus for continuous agitation and mixing of the culture. The reactor with medium is steamed at 100° C. for about 60 minutes in order to sterilize the reactor and the growth medium.

The reactor is then allowed to cool down. Once the reactor reaches about 35° C., antibiotics are added to the medium to prevent bacterial contamination. The antibiotic composition comprises 300 g streptomycin and 20 g oxytetracycline dissolved in 4L DI water. Other reactor tubing and openings are sprayed with isopropyl alcohol (IPA) to sterilize them. Small-scale reactors are used for growing Starmerella bombicola inoculum cultures. The culture is grown for at least 42 to 48 hours at 26 to 28° C. in the small-scale reactors.

Once the stainless-steel fermentation reactor reaches 30° C., it is then inoculated with about 25L of the inoculum culture.

The temperature of fermentation is held at 23 to 28° C. After about 22 to 26 hours, the pH of the culture is set to about 3.0 to 4.0, or about 3.5, using 20% NaOH. The fermentation reactor comprises a computer that monitors the pH and controls the pump used to administer the base, so that the pH remains at 3.5.

After about 1-10 days of cultivation (120 hours +/−1 hour), if 7.5 ml of a SLP layer is visible with no oil visible and no glucose detected, the batch is ready for harvesting.

The culture is harvested to a first collection container and left undisturbed for at least 24 hours. A layer of SLP settles to the bottom of the first collection container. The settled SLP layer is harvested to a second collection container, leaving behind the top layer of cells and supernatant. The cells and supernatant comprise the yeast fermentation product.

### Example 3

### Feeding Animals

In one embodiment, the yeast fermentation product can be useful as a nutritional supplement and/or digestive aide for animals, such as livestock, fish and pets.

In one embodiment, methods are provided for feeding an animal, wherein a yeast fermentation product of the subject invention is applied to the animal's food and/or drinking water, and wherein the animal ingests the food and/or water with the yeast fermentation product therein.

Additionally, the methods can be useful for providing a nutrient to an animal, as well as for enhancing the digestive and immune health of an animal.

Due to the presence of, for example, biosurfactants in the yeast fermentation product, the methods can enhance nutrient absorption through the animal's digestive tract. Additionally, the methods can enhance the immune health of an animal and reduce the need for antibiotics due to the antimicrobial properties of the product.

As used herein, “livestock” refers to any domesticated animal raised in an agricultural or industrial setting to produce commodities such as food, fiber and labor. Types of animals included in the term livestock can include, but are not limited to, alpacas, llamas, beef and dairy cattle, bison, pigs, sheep, goats, dogs, horses, mules, asses, camels, chickens, turkeys, ducks, geese, guinea fowl, and squabs.

As used herein, “pets” can include domesticated animals that are raised and cared for by a human for protection and/or companionship, such as, for example, dogs, cats, birds, rodents, and reptiles.

In one embodiment, the yeast fermentation product can be applied to the animal's food, and/or drinking water as a nutritional supplement and/or digestive aide. The product can be introduced into the food and/or water as a liquid composition by pouring and/or mixing, and the animal then ingests the supplemented food and/or water.

In one embodiment, the composition can be mixed in with standard feed components and formulated into uniform, homogenized pellets. The supplemented feed pellet can comprise consistent concentrations of the microbe-based composition per pellet. Methods known in the art for producing feed pellets can be used, including pressurized milling. Preferably, the pelleting process is “cold” pelleting, or a process that does not use high heat or steam.

The yeast fermentation products may be further treated to facilitate rumen bypass. The product may be spray-dried, and optionally treated to modulate rumen bypass, and added to feed as a nutritional source.

As a food supplement and/or digestive aide, the yeast fermentation products can provide, among other benefits, sources of amino acids (including essential amino acids), peptides, proteins, vitamins, microelements, fats, fatty acids, lipids such as phospholipid, carbohydrates, sterols, enzymes, and trace minerals such as, iron, copper, zinc, manganese, cobalt, iodine, selenium, molybdenum, nickel, fluorine, vanadium, tin and silicon.

In certain embodiments, the yeast fermentation product can be applied to a fish's environment, such as the water of a fish farm, in the form of, for example, a liquid solution, or as a dry powder, a meal, or feed flakes or pellets.

In one embodiment, particularly for use in aquatic settings, the yeast fermentation product comprises inactive yeasts. The composition is prepared by cultivating the desired microorganism, inactivating the microbe by micro-fluidizing (or by any other method known in the art not to cause protein denaturation), pasteurizing and adding it to the food stuff in concentrated form. In one embodiment, inactivation occurs at pasteurization temperature (up to 65° to 70° C. for a time period sufficient to inactivate 100% of the yeast cells) and increasing pH value up to about 10.0. This induces partial hydrolysis of cells and allows for freeing of some nutritional components therein. Then, the composition is neutralized to a pH of about 7.0-7.5 and the various components of hydrolysis are mixed. The resulting microbe-based product can then be used for, for example, fish feed and treatment of fish farm water.

### Example 4

### Bioremediation

In one exemplary embodiment, the yeast fermentation product can be used for bioremediation of soils, surfaces, waters, or other sites that have contaminants thereon.

In one embodiment, methods are provided for remediating a site having a contaminant thereon, wherein a yeast fermentation product of the subject invention is applied to the site. In certain embodiments, the site is soil, a surface or water that has been contaminated with a hydrocarbon, e.g., from an oil spill, or another contaminant, such as arsenic. The method can further comprise applying one or more growth-promoting substances to the site to encourage the yeast in the composition to grow and produce biosurfactants at the site.

Bioremediation can include both in situ and ex situ bioremediation methods of contaminated solids, soils, and waters (ground and surface) wherein in situ techniques are defined as those that are applied to, for example, soil and groundwater at the site with minimal disturbance. Ex situ techniques are those that are applied to, for example, soil and groundwater that have been removed from the site via, for example, excavation (soil) or pumping (water).

In some embodiments of the present invention, an in situ technique involves mechanically spreading a remediation composition of the present invention onto the contaminated surface. This may be performed using a standard spreader or sprayer device. In some embodiments, a single spreading step may complete the application process, wherein all of the components are included in a single formulation. In other embodiments, which use two- or multiple-part formulations, multiple spreading steps may be used. In one embodiment, the bioremediation composition may be rubbed, brushed, or worked into the surface or ground to be cleaned using a mechanical action to work the bioremediation composition into the pores or grains of the surface and/or to spread the bioremediation composition around the contaminated area. In still further embodiments, when applied to solid surfaces, the application of a remediation composition may be subsequently followed by application of a liquid, such as water. The water may be applied as a spray, using standard methods known to one of ordinary skill in the art. Other liquid wetting agents and wetting formulations may also be used. Some embodiments of the present invention include the infiltration of water-containing nutrients and oxygen or other electron acceptors for groundwater treatment, after application of the solid or liquid bioremediation composition of the present invention.

Ex situ techniques typically involve the excavation or removal of contaminated soil from the ground. Examples of ex situ bioremediation techniques that may be used in some embodiments of the present invention include land-farming, composting, biopiles, and bioreactors.

In one embodiment the microbial composition of the subject invention is dispersed in oil-contaminated soil while being supported on a carrier. The carrier can be made of materials that can retain microorganisms thereon relatively mildly and thus allow easy release of microorganisms thus proliferated. The carrier is preferably inexpensive and can act as a nutrient source for the microorganisms thus applied, particularly a nutrient source that can be gradually released. Preferred biodegradable carrier materials include cornhusk, sugar industry waste, or any agricultural waste. The water content of the carrier typically varies from 1% to 99% by weight, preferably from 5% to 90% by weight, more preferably from 10% to 85% by weight. When the water content of the carrier is too low, microorganism survival is difficult. On the other hand, when the water content of the carrier is too high, the resulting carrier exhibits a deteriorated physical strength that makes itself difficult to handle.

In certain preferred embodiments, the yeast fermentation product, is applied to a target remediation site alongside substances that encourage the microorganisms in the product to grow at the site and, ideally, produce more biosurfactants there. These growth-promoting substances include, but are not limited to, carbon sources (e.g., molasses, glycerol), inorganic/organic nitrogen sources, lipids, proteins, or any other known growth-promoting nutrients.

### Example 4

### Mining

In one exemplary embodiment, the yeast fermentation product can be used for recovering valuable minerals from ore and/or coal.

Thus, in one embodiment, methods are provided for recovering valuable minerals from ore and/or coal, wherein the valuable minerals are gold, silver, copper, cobalt, nickel, zinc, or others described herein.

As used herein, “ore” refers to a naturally occurring solid material from which a valuable mineral and/or metal can be profitably extracted. Ores are often mined from ore deposits, which comprise ore minerals containing the valuable substance. “Gangue” minerals are minerals that occur in the deposit but do not contain the valuable substance.

In certain embodiments, the subject invention provides a method for extracting valuable minerals and/or metals from ore, wherein the method comprises obtaining ore from an ore deposit, said ore comprising one or more valuable minerals and/or metals (in addition to gangue, or less valuable ore minerals); applying the yeast fermentation product to the ore; allowing the valuable minerals and/or metals to separate from the ore gangue; and collecting the valuable minerals and/or metals.

In some embodiments, the method can be used for bioleaching. In one embodiment, the ore can be unmined, wherein the ore can be sprayed with the yeast fermentation product while it is still located in an ore deposit or mine. In one embodiment, the ore can be mined and, optionally, crushed or ground into smaller particles prior to being sprayed with the yeast fermentation product.

The yeast fermentation product can enhance recovery of valuable minerals and/or metals from ore due to, for example, the microbial production of biosurfactants, solvents, enzymes, proteins, peptides and amino acids that allow the microbes to sequester nanoparticles of the minerals and/or metals from the ore.

As used herein, the terms “valuable minerals” and “valuable metals” refer to any mineral or metal, respectively, that is extracted or mined from the earth, which has some economic value. The value of the mineral and/or metal is typically measured by how abundant or rare it is, with rarer minerals and/or metals having a higher economic value per unit of weight over those that are more abundant.

Examples of valuable metals and/or elements that can also be extracted using the subject invention, as well as valuable minerals that produce and/or comprise those metals and/or elements, include but are not limited to cobalt (e.g., erythrite, skytterudite, cobaltite, carrollite, linnaeite, and asbolite (asbolane)); copper (e.g., chalcopyrite, chalcocite, bornite, djurleite, malachite, azurite, chrysocolla, cuprite, tenorite, native copper and brochantite); gold (e.g., native gold, electrum, tellurides, calaverite, sylvanite and petzite); silver (e.g., sulfide argentite, sulfide acanthite, native silver, sulfosalts, pyrargyrite, proustite, cerargyrite, tetrahedrites); aluminum (e.g, bauxite, gibbsite, bohmeite, diaspore); antimony (e.g., stibnite); barium (e.g., barite, witherite); cesium (e.g., pollucite); chromium (e.g., chromite); iron (e.g., hematite, magnetite, pyrite, pyrrhotite, goethite, siderite); lead (e.g., galena, cerussite, anglesite); lithium (e.g., pegmatite, spodumene, lepidolite, petalite, amblygonite, lithium carbonate); magnesium (e.g., dolomite, magnesite, brucite, carnallite, olivine); manganese (e.g., hausmannite, pyrolusite, barunite, manganite, rhodochrosite); mercury (e.g., cinnabar); molybdenum (e.g., molybdenite); nickel (e.g., pentlandite, pyrrhotite, garnierite); phosphorus (e.g., hydroxylapatite, fluorapatite, chlorapatite); platinum group (platinum, osmium, rhodium, ruthenium, palladium) (e.g., native elements or alloys of platinum group members, sperrylite); potassium (e.g., sylvite, langbeinite); rare earth elements (cerium, dysprosium, erbium, europium, gadolinium, holmium, lanthanium, lutetium, neodymium, praseodymium, samarium, scandium, terbium, thulium, ytterbium, yttrium) (e.g., bastnasite, monazite, loparite); sodium (e.g., halite, soda ash); strontium (e.g., celestite, strontianite); sulfur (e.g., native sulfur, pyrite); tin (e.g., cassiterite); titanium (e.g., scheelite, huebnerite-ferberite); uranium (e.g., uraninite, pitchblende, coffinite, carnotite, autunite); vanadium; zinc (e.g., sphalerite, zinc sulfide, smithsonite, hemimorphite); and zirconium (e.g., zircon).

Additional elements and/or minerals include, e.g., arsenic, bertrandite, bismuthinite, borax, colemanite, kernite, ulexite, sphalerite, halite, gallium, germanium, hafnium, indium, iodine, columbite, tantalite-columbite, rubidium, quartz, diamonds, garnets (almandine, pyrope and andradite), corundum, barite, calcite, clays, feldspars (e.g., orthoclase, microcline, albite); gemstones (e.g., diamonds, rubies, sapphires, emeralds, aquamarine, kunzite); gypsum; perlite; sodium carbonate; zeolites; chabazite; clinoptilolite; mordenite; wollastonite; vermiculite; talc; pyrophyllite; graphite; kyanite; andalusite; muscovite; phlogopite; menatite; magnetite; dolomite; ilmenite; wolframite; beryllium; tellurium; bismuth; technetium; potash; rock salt; sodium chloride; sodium sulfate; nahcolite; niobium; tantalum and any combination of such substances or compounds containing such substances.

### Example 5

### Argriculture

In one exemplary embodiment, the yeast fermentation product can be used for enhanced agricultural production, wherein the target application site is soil, a plant and/or a seed.

In one embodiment, methods are provided for enhancing production in agriculture, wherein the yeast fermentation product is applied to a plant and/or its surrounding environment. The product can be applied to a plant or to the soil in which the plant grows through the irrigation system, or it can be poured or sprinkled onto the soil or plant in the form of a dried product. The product can also be applied to a plant seed prior to planting the seed in soil.

In one embodiment, the subject invention provides a method of improving plant health and/or increasing crop yield by applying the yeast fermentation product to soil, seeds, or directly to a plant or plant parts (e.g., roots, leaves).

The methods can also be used to control a pest that affects the plant. Furthermore The yeast fermentation product can serve as a replacement for water when irrigating a plant, and/or as a soil wetting agent.

As used herein, “enhancing” means improving or increasing. For example, enhanced plant health means improving the plant's ability grow and thrive, including the plant's ability to ward off pests and/or diseases, and the plant's vigor, vitality and ability to survive environmental stressors (e.g., droughts and/or overwatering). Enhanced plant growth means increasing the size and/or mass of a plant (e.g., increased canopy, height, trunk caliper, branch length, and/or overall growth index), and/or improving the ability of the plant to reach a desired size and/or mass. Enhanced yields mean improving the end products produced by the plants in a crop, for example, by increasing the number of fruits per plant, increasing the size of the fruits, and/or improving the quality of the fruits (e.g., taste, texture and/or color).

In one embodiment, additional agents, such as prebiotics, proteins, natural or commercial fertilizers, natural or commercial pesticides or purified biosurfactants can be applied with the yeast fermentation product. Examples of prebiotics include, e.g., kelp extract, folic acid, folate, malic acid, fulvic acid, humate and/or humic acid.

In specific embodiments, the yeast fermentation product can be used for promoting crop vitality; enhancing crop yields; enhancing plant immune responses; enhancing insect, pest and disease resistance; controlling insects, nematodes, diseases and weeds; improving plant nutrition; improving the nutritional content of agricultural and forestry and pasture soils; and promoting improved and more efficient water use.

### Example 6

### Oil and Gas Recovery

In one exemplary embodiment, the yeast fermentation product can be used for enhanced oil recovery (EOR), wherein the target application site is an oil and/or gas well, a subterranean formation, and/or any piece of equipment associated with oil and/or gas recovery, transport, refining, and processing.

In one embodiment, methods are provided for increasing oil production, wherein the yeast fermentation product is applied to an oil and/or gas well, a subterranean formation, and/or to a piece of equipment associated with oil and/or gas recovery, transport, refining and/or processing.

Combined with the characteristics of low toxicity and biodegradability, the biosurfactants in the yeast fermentation product are advantageous for use in the oil and gas industry for a wide variety of petroleum industry applications. These applications include, but are not limited to, stimulation of oil and gas wells (to improve the flow of oil into the well bore); removal of contaminants and/or obstructions such as paraffins, asphaltenes and scale from equipment such as rods, tubing, liners, tanks and pumps; prevention of the corrosion of oil and gas production and transportation equipment; reduction of H₂S concentration in crude oil and natural gas; reduction in viscosity of crude oil; upgradation of heavy crude oils and asphaltenes into lighter hydrocarbon fractions; cleaning of tanks, flowlines and pipelines; enhancing the mobility of oil during water flooding though selective and non-selective plugging; and fracturing fluids.

When used in oil and gas applications, the yeast fermentation product of the present invention can be used to lower the cost of microbial-based oilfield compositions and can be used in combination with other chemical enhancers, such as polymers, solvents (e.g., isopropyl alcohol, terpenes), salts (e.g., ammonium phosphate), chelating agents (e.g., EDTA, citric acid, sodium citrate), fracking sand and beads, emulsifiers, surfactants, and other materials known in the art.

### Example

### Human Health

In one embodiment, the yeast fermentation product can be used as an adjuvant composition and/or as a digestive aide in humans, wherein the target site is a health-promoting substance, such as a pharmaceutical, a supplement, a nutrient and/or water, that is consumed by and/or administered to a human.

In one embodiment, methods are provided for increasing the bioavailability of a pharmaceutical, a supplement, a nutrient and/or of water, wherein a yeast fermentation product of the subject invention is applied to the pharmaceutical, supplement, nutrient and/or water prior to the pharmaceutical, supplement, nutrient and/or water being consumed by and/or administered to the human.

Preferably, the composition is dried and the concentration of hiosurfactant in the composition is tested and standardized according to safety standards prior to being consumed by and/or administered to a human.

The yeast fermentation product can be formulated into a health-promoting substance, or applied to a human alongside a health-promoting substance, to improve the bioavailability of the substance to the human. In certain specific embodiments, the yeast-based product can aid in suppressing P-glycoproteins and/or modulating other physical barrier mechanisms that would otherwise reduce the penetration of certain substances into, for example, epithelial cells and/or across the blood-brain barrier.

The health-promoting compound can be, for example, a pharmaceutical compound, a nutritional supplement, or even simply water. In one embodiment, the subject compositions are formulated as an orally-consumable product, such as, for example, a capsule, a pill or a drinkable liquid. In another embodiment, the subject compositions are formulated to be administered via injection, suppository, inhalation, subcutaneously, cutaneously, or any other mode of administration known in the medical arts.

In certain embodiments, the yeast fermentation product can also serve as a nutritional supplement and/or a digestive aide for a human, providing, among other benefits, sources of amino acids (including essential amino acids), peptides, proteins, vitamins, microelements, fats, fatty acids, lipids such as phospholipid, carbohydrates, sterols, enzymes, and trace minerals such as, iron, copper, zinc, manganese, cobalt, iodine, selenium, molybdenum, nickel, fluorine, vanadium, tin and silicon, when ingested by the human.

1. A composition, produced by:
cultivating a biosurfactant-producing yeast in culture medium using a submerged fermentation system, where said yeast produces a biosurfactant into the medium; and
harvesting the biosurfactant to leave behind a supernatant and yeast cell biomass in the fermentation system,
wherein the composition comprises the supernatant and yeast cell biomass.

2-3. (canceled)

4. The composition of claim 3, wherein the composition comprises about 1-4 g/L of residual biosurfactants.

5. The composition of claim 1, wherein the yeast is Starmerella bombicola.

6. The composition of claim 1, wherein the biosurfactant is a sophorolipid (SLP).

7. (canceled)

8. A method for feeding an animal, wherein a composition of claim 1 is applied to the animal's food and/or drinking water prior to being ingested by the animal.

9. The method of claim 8, wherein the composition is poured, in liquid form, into the animal's food and/or water.

10. The method of claim 8, wherein the composition is formulated with the animal's food.

11. The method of claim 10, wherein the composition is formulated into feed pellets.

12. A method for remediating a site having a contaminant thereon, wherein a composition of claim 2 is applied to the site.

13. The method of claim 12, wherein the site is soil, a surface or water that has been contaminated with a hydrocarbon.

14-15. (canceled)

16. A method for recovering a mineral from ore or coal, wherein a composition of claim 1 is applied to the ore or coal.

17. The method of claim 16, wherein the mineral is gold, copper, cobalt, nickel, or silver.

18. A method for enhancing production in agriculture, wherein a composition of claim 1 is applied to a plant and/or its surrounding environment.

19. The method of claim 18, used to enhance the growth, health and/or yield of the plant.

20. The method of claim 18, wherein the composition is applied to soil in which the plant grows.

21. The method of claim 18, wherein the composition is applied directly to the plant.

22. The method of claim 18, used to control a pest that affects the plant.

23. The method of claim 18, wherein the composition is applied to a plant seed prior to planting the seed in soil.

24. (canceled)

25. A method of enhancing oil recovery, wherein a composition of claim 1 is applied to an oil and/or gas well, a subterranean formation, and/or to a piece of equipment associated with oil and/or gas recovery, transport, refining and/or processing.

26. A method for increasing the bioavailability of a pharmaceutical, a supplement, a nutrient and/or of water, wherein a composition of claim 1 is applied to the pharmaceutical, supplement, nutrient and/or water prior to the pharmaceutical, supplement, nutrient and/or water being consumed by and/or administered to subject.
